(19) United States
(12) Patent Application Publication
(10) Pub. No.: US 2026/0140078 A1
(43) Pub. Date: May 21, 2026

(54) METHOD FOR EVALUATING SILICON SINGLE CRYSTAL INGOT

(71) Applicant: GLOBALWAFERS JAPAN CO., LTD., Kitakanbara-gun, Niigata (JP)

(72) Inventors: Kei Watanabe, Kitakanbara-gun, Niigata (JP); Hiroyuki Tsubota, Kitakanbara-gun, Niigata (JP)

(73) Assignee: GLOBALWAFERS JAPAN CO., LTD., Kitakanbara-gun, Niigata (JP)

(21) Appl. No.: 18/994,945

(22) PCT Filed: Feb. 14, 2023
(86) PCT No.: PCT/JP2023/004977

§ 371 Date: Jan. 15, 2025

(30) Foreign Application Priority Data
Jul. 29, 2022 (JP) 2022-122039

(51) Int. Cl. G01N 27/04; C30B 15/04; C30B 29/06; G01N 33/00

(52) U.S. Cl. CPC G01N 27/041; G01N 33/39; C30B 15/04; C30B 29/06

(57) ABSTRACT

A method for evaluating a silicon single-crystal ingot is provided that reduces the time required to evaluate the resistivity variation in the crystal length direction and improves wafer yield. In the method for evaluating a silicon single-crystal ingot according to the present embodiment, a counter doping is performed by adding a sub-dopant during the pulling of silicon single-crystals ingot by the CZ method, resistivity on a side surface in a crystal length direction is measured by a four-point probe method in a silicon single-crystal ingot 1 state after the silicon single-crystal ingot is pulled up, and a reject range is determined on the basis of a resistivity distribution in the crystal length direction.

### FIELD OF TECHNOLOGY

This invention relates to a method for evaluating silicon single-crystal ingots produced by the Czochralski method (CZ method).

### BACKGROUND TECHNOLOGY

In the CZ method, silicon single-crystal ingots (hereinafter referred to as “single-crystal ingots”) are produced by bringing a seed crystal into contact with silicon melt and pulling the seed crystal slowly while rotating it. The CZ method is commonly used to produce large-diameter single-crystal ingots.

Meanwhile, it is known that the resistivity of single-crystal ingots produced by the CZ method varies along the crystal growth direction (crystal length direction). In recent years, due to the quality requirements of semiconductor wafers (hereinafter referred to as wafers) produced from single-crystal ingots, it has become important to keep the resistivity within a desired range.

For example, when measuring the resistivity of a wafer, conventionally, a single-crystal ingot grown by the CZ method is peripherally ground to a predetermined size (diameter), the part of the head and tail cone that cannot be used as a product is cut off, the resulting single-crystal ingot is cut at a predetermined position and made into a block of a length that can fit into a slicing device such as an inner diameter saw or wire saw. At this point, sample wafers for testing resistivity and other parameters are simultaneously cut out. The cut sample wafers can then be used to measure resistivity. Each block is then sliced into wafers of a predetermined thickness. The sliced wafers are then removed and the resistivity is measured in the crystal length direction.

When growing single-crystal ingots by the CZ method, there is a phenomenon in which the resistivity changes in the direction of crystal growth when dopants are added. This is due to the segregation of dopants, and as the silicon melt in the crucible decreases with single-crystal growth, the concentration of dopants in the residual liquid gradually increases, and the resistivity of the single-crystal continuously decreases. The segregation coefficient of phosphorus P is 0.35, which is lower than that of boron B, 0.8, which is widely used as a dopant in p-type crystals, and the decrease in resistivity from the top to the bottom is more pronounced than in p-type crystals. Therefore, a problem arises that the usable portion becomes small as the product and it is difficult to improve the yield.

Furthermore, as a countermeasure to the problem described above of the resistivity deviating from the desired resistivity range in the crystal length direction, a method of measuring the resistivity in the state of a single crystal ingot is disclosed in Patent Literature 1 (PTL 1) below.

Specifically, the resistivity in the crystal length direction is measured on the side surface of the single-crystal ingot, the desired resistivity position at which the desired resistivity is obtained is specified, a block of a predetermined length is cut, and wafers are sliced from the block. The resistivity measurement method along the crystal length direction uses the resistivity measurement method by the four-point probe method. This makes it possible to produce wafers with a resistivity of 1 Ω·cm or less.

It is known that it is difficult to achieve uniform resistivity distribution along the crystal length direction in single-crystal ingots grown by the CZ method due to segregation. For this reason, single-crystal ingots have conventionally been grown by the so-called counter doping method, in which a main dopant and a sub-dopant of the opposite conductivity type are added (see Patent Literature 2 below).

According to the patent literature 2 below, the counter doping consists of the process of doping the main dopant, (e.g., phosphorus), which causes an n-type conductivity type, and continuously or intermittently doping additional sub-dopant (e.g., boron), which causes a p-type conductivity type opposite to the n-type conductivity type, while growing the single-crystal ingot, in accordance with the solidification rate expressed as (crystallized weight)/(initial weight of silicon raw material). In some cases, a single-crystal grown by the counter doping is hereinafter referred to as a counter doped crystal.

### CITATION LIST

### Patent Literature

PTL 1: JP-A-2012-129308
PTL 2: JP-A-2016-060667

### SUMMARY OF INVENTION

### Technical Problem

According to PTL 1, the yield of wafers can be improved because a block can be sliced to include more of the portion having the desired resistivity when producing wafers having a low resistivity of less than 1 Ω·cm or less. In addition, because the resistivity is measured in a single-crystal ingot state, the time required for resistivity evaluation can be significantly reduced compared to the method in which the resistivity is measured after slicing a sample wafer for testing.

The production method disclosed in PTL 1 is problematic in the following.

For example, in the case of heavily doped (low resistivity) wafers of 1 Ω·cm or less, since the dopant concentration of the main dopant is sufficiently high compared to the thermal donor, and the effects of thermal donors are less even without heat treatment, the resistivity can be measured as the single-crystal ingot is. In contrast, in the case of a lightly doped crystal with a desired resistivity of, for example, 10 Ω·cm or more, since it is affected by the thermal donor and the donor killer process of heat treatment is necessary to achieve the desired resistivity, but the donor killer process is difficult to perform in the single-crystal ingot state. That is, in the case of a lightly doped crystal, it is difficult to evaluate the true resistivity, hereafter referred to as the true resistivity, in the crystal length direction from which the effect of the thermal donor is excluded, even when the resistivity is measured in the single-crystal ingot state.

Meanwhile, a counter doped crystal has a region where the resistivity increases rapidly and then decreases rapidly (hereafter referred to as the high resistivity region) near a position in the crystal length where a sub-dopant is added. Thus, the high resistivity region should be detected in the counter doped crystal described above.

The present invention is made in consideration of the above problem, and the object thereof is to provide an evaluation method that enables the detection of the high-resistivity region in the crystal-length direction in the single-crystal ingot state in a lightly counter doped crystal.

### Solution to Problem

The method according to the present invention is for evaluating a single-crystal ingot that has been counter doped by adding a sub-dopant during the pulling of silicon single-crystals by the CZ method. The method includes a step of measuring the resistivity in a crystal length direction by a four-point probe method on a side surface of a single-crystal ingot in a single-crystal ingot state, and a step of determining a reject range of the single-crystal ingot (non-product range), on the basis of a resistivity distribution in the crystal length direction.

In the single-crystal ingot, there is a region in which a predetermined threshold or less of resistivity variation is stable, (e.g., 1 to 3%), hereinafter referred to as a stable region. In the counter doped crystal according to the present invention, the reject range is determined with reference to a position, hereinafter also referred to as a reference position, at which there is a peak value of resistivity higher than 10% or more compared to the resistivity of the stable region of the head side. That is, the reject range is defined as a range between positions that are separated from the peak position (the reference position is defined as the position where the resistivity exceeds 10% for the first time when there are multiple data exceeding 10%) to the head side and the tail side by predetermined lengths, for example, 8 to 12 mm toward the head and 10 to 20 mm toward the tail, respectively. The reject range on the tail side may be up to the position where the resistivity variation in the crystal length direction becomes below a threshold (e.g., 1% or less) and starts to depend on segregation (hereinafter referred to as the stable position).

With the above configuration, a high-resistivity region due to counter doping can be specified from the resistivity distribution on the side surface of the single-crystal ingot, the reject range is determined in the single-crystal ingot state, and the yield of wafers can be significantly increased compared with a method (conventional method) in which the resistivity is evaluated with the test sample wafers cut from the single-crystal ingot. In addition, the time required for resistivity evaluation can be shortened compared with the conventional method.

In the evaluation method of the single-crystal ingot according to the present invention, the measurement range of the resistivity desirably includes at least a range from the sub-dopant added position to the position where the resistivity becomes the desired resistivity again stable after the resistivity increases and rapidly decreases.

Furthermore, in the evaluation method of the single-crystal ingot according to the present invention, the resistivity of the single-crystal ingot should be 10 Ω·cm or more.

### Advantageous Effects of Invention

With the present invention, the time required for evaluating the resistivity in the crystal length direction can be shortened, and in addition, the yield of wafers can be improved.

### BRIEF DESCRIPTION OF DRAWINGS

FIG. 1 is a viewgraph showing the outline of the measuring the resistivity on the side surface of a single-crystal ingot;

FIG. 2 is a viewgraph showing the single-crystal ingot of an example;

FIGS. 3A and 3B are viewgraphs showing the measurement results of the resistivity on the side surface of a single-crystal ingot;

FIGS. 4A and 4B are viewgraphs showing a reject range;

FIGS. 5A and 5B are viewgraphs showing the resistivity measurement results at the center and the outer circumference of a wafer;

FIGS. 6A to 6D are viewgraphs showing the resistivity measurement results in the radial direction of the wafer.

### DESCRIPTION OF EMBODIMENTS

An embodiment of the method of evaluating the resistivity of a single-crystal ingot will be described below. The present invention is not limited to the embodiments described above. Although the embodiment is described using an n-type single-crystal ingot, it is not limited thereto but may be applied to a p-type single-crystal ingot.

In this embodiment, when lightly-doped (10 Ω·cm or more) n-type single-crystal ingots are grown by the CZ method, counter doping is performed by adding a p-type dopant as a sub-dopant during the pulling. For example, it is difficult to achieve a uniform resistivity distribution in the crystal growth direction (crystal length direction) for single-crystal ingots grown by the CZ method due to segregation, but this problem can be solved by counter doping with a sub-dopant of the opposite polarity to the main dopant.

For example, when silicon crystallizes, the concentration of the dopant introduced into the crystal is lower than the concentration of the dopant in the melt. As the single-crystal ingot grows continuously, more dopant remains in the melt and the concentration of the dopant in the melt gradually increases. With this, the concentration of the dopant in the crystal gradually increases, and the resistivity decreases. To prevent the resistivity from falling out of the desired resistivity range due to the decrease in the resistivity, the sub-dopant of opposite polarity is added to the melt in the appropriate amount (counter doping). The resistivity increases at the position of the crystal length where the sub-dopant is added, and then the resistivity decreases and stabilizes depending on the segregation.

In the present embodiment, for example, phosphorus (P), arsenic (As), and antimony (Sb) can be used as an n-type dopant (for the main dopant), and for example, boron (B), aluminum (Al), and gallium (Ga) can be used as a p-type dopant (for the sub-dopant). They can be used when the main dopant is p-type and the sub-dopant is n-type.

In the present embodiment, after a counter doped crystal is pulled, subjected to peripheral grinding to the predetermined dimension (diameter), and the head and tail cone portions are cut out, then, the resistivity in the crystal length direction is measured by the four-point probe method on the side surface in the single-crystal ingot state and the relative resistivity variation in the crystal length direction is evaluated based on the resistivity distribution. Specifically, based on the resistivity distribution in the crystal length direction (relative resistivity distribution), a range is detected from a position that is backward toward the head side by a predetermined length (e.g., 8 to 12 mm) from the peak position (reference position) where the resistivity variation is higher resistivity than 10% or more compared to the resistivity of the stable region of the head side to the stable position. Alternatively, a reject area is determined by the length between the position that is a predetermined length (e.g., 8 to 12 mm) back toward the head side from the reference position and the position that is a predetermined length (e.g., 10 to 20 mm) forward toward the tail side from the reference position. Note that the single-crystal ingot is not necessarily peripherally ground.

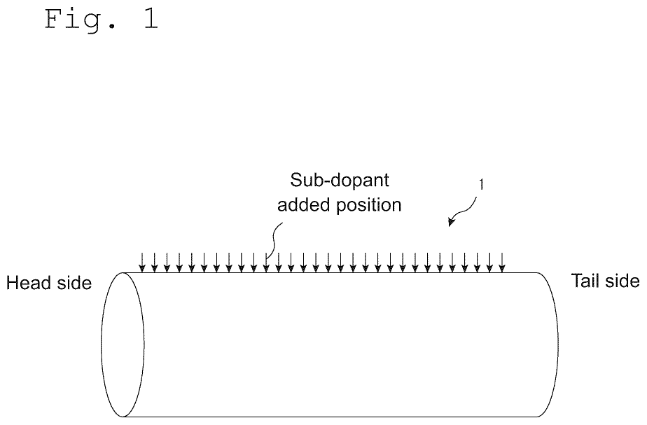

FIG. 1 is a viewgraph showing the outline of the measuring the resistivity on the side surface of a single-crystal ingot. After the counter doped crystal is pulled up, a single-crystal ingot 1 shown in FIG. 1 is obtained by peripherally grinding the counter doped crystal and cutting the head and the tail cone thereof. In addition, since the positions in the crystal length direction where the sub-dopant is added (sub-dopant added positions) are recorded in advance when the crystal is pulled up, the resistivity on the side surface of the single-crystal ingot 1 including the sub-dopant added positions is measured by the four-point probe method. Specifically, as shown in FIG. 1, the resistivity in the side surface of the single-crystal ingot 1 at thirty (30) points along the crystal growth direction by an interval of 1 mm as a starting point which is the position 10 mm backward toward the head side from the sub-dopant added position. At this time, the resistivity increases sharply near the sub-dopant added position and then decreases sharply, and the resistivity gradually decreases depending on the segregation. The resistivity measurement range should include only a range from the sub-dopant added position to the position where the resistivity stabilizes again to a desired resistivity, after increasing, and may not be limited to the thirty points at 1 mm intervals.

In the present embodiment, the resistivity is measured from the stable region on the head side in the crystal length direction, and the reference position is defined by a peak position where the peak value of the resistivity becomes not less than 10% higher than that of the stable region. In addition, the stable position is denoted as the position where the resistivity variation rate on the tail side of the crystal length direction stabilizes and the resistivity variation rate becomes below the threshold (e.g., 1% to 3%) and the resistivity starts to depend on the segregation, and let the reject range be a range from a position 10 mm backward toward the head side of the reference position to the stable position. As a result, the processing loss in the wafer machining process and the time required to evaluate the resistivity variation can be significantly reduced compared to the method of slicing test sample wafers from a single-crystal ingot and measuring their resistivity (conventional method). Note that the reject range on the tail side from the peak position is not limited to the range up to the stable position and may be a predetermined length. For example, the reject range on the tail side may be 10 mm or more and not more than 20 mm from the reference position. The reject range on the head side from the reference position may be 8 mm or more and not more than 12 mm from the peak position (reference position).

In an n-type counter doped crystal grown by the CZ method as described above, a certain amount of oxygen is dissolved by the above production method; the dissolved oxygen partially becomes a thermal donor that acts as an n-type dopant and reduces resistivity. By subjecting the counter doped crystal grown by the CZ method to a heat treatment (a donor killing process), the original resistivity of the crystal determined by the main dopant can be obtained. Thermal donors are generated in large numbers when the thermal history at around 450° C. is long and the oxygen concentration is high during the crystal growth.

In addition, the resistivity peaks formed by the resistivity variation on the side surface of the single-crystal ingot 1 vary depending on the dopant concentration, the number of the thermal donors, and the amount of the variation of the thermal donor at the measurement position. In the present embodiment, when the resistivity is 10 Ω·cm or more regardless of the oxygen concentration, peaks with a resistivity variation of 10% or more are formed near the position of the crystal length direction where the sub-dopant is added.

The resistivity variation on the side surface of the single-crystal ingot 1 due to the thermal donors can be represented by the following equation:

$$Resistivity variation due to thermal donors = ( resistivity corresponding to the amount of thermal donor variation ) / ( resistivity of single - crystal ingot + resistivity due to thermal donor amount ) .$$

For example, when the oxygen concentration in the single-crystal ingot 1 is high, since the resistivity on the side surface is substantially determined by the number of thermal donors and the denominator becomes small due to the effect of the thermal donors, the ratio (the resistivity variation) becomes large. On the other hand, when the oxygen concentration is low, because the amount of thermal donor variation becomes small (the resistivity due to the thermal donors increases), the numerator becomes large. Thus, a peak is formed where the resistivity increases by 10% or more than that of the stable region because of the changes in the denominator or numerator due to the variation of the oxygen concentration, and the reference position (the position where the resistivity variation is large by counter doping) can be detected.

Note that the resistivity is measured by the four-point probe method in the present embodiment, but the method is not limited thereto; any measurement method can be used as long as the resistivity is measured in the crystal length direction on the side surface in the state of single-crystal ingot state.

### Effects

As described above, the time required to evaluate the resistivity can be reduced in the evaluation method of a single-crystal ingot in the present embodiment.

### EXAMPLES

Next, examples of the evaluation method of silicon single-crystal ingot according to the present invention will be described. It should be noted that the present invention is not limited to the examples described below.

### Example 1

A lightly doped n-type counter doped crystal was pulled up by counter doping where the main dopant was phosphorus and the sub-dopant was boron. At this time, the sub-dopant boron was added at positions of 500 mm and 800 mm of the crystal length during the growth of a counter doped crystal under the conditions of a crystal rotation speed of 10 rpm, a crucible rotation speed of 1 rpm, a pulling speed of 1 mm/min, a magnetic field strength of 2000 G. In Example 1, the desired resistivity was 50 Ω·cm (phosphorus concentration: about 8.64E13/cm³) and the desired oxygen concentration was 0.55E18/cm³.

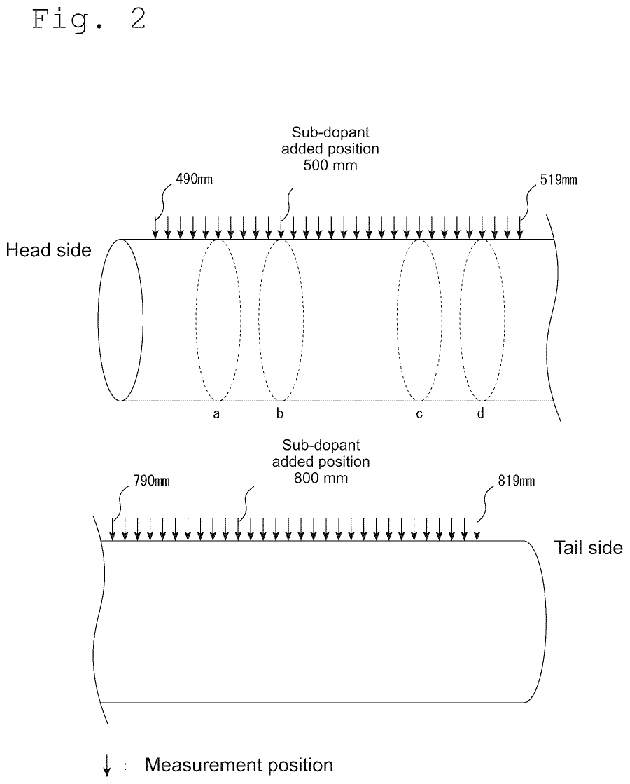

Using the above settings, a counter doped crystal was pulled up and was finished to a diameter of 300 mm by peripheral grinding, the head and tail cones were cut off, and a single-crystal ingot was produced as shown in FIG. 2.

Then, the resistivity in the crystal length direction on the side surface in the state of the single-crystal ingot state was measured using the four-point probe method. Specifically, the resistivity of the side surface of the single-crystal ingot was measured at 30 points with a separation of 1 mm along the crystal growth length, starting from the position 10 mm back from the sub-dopant added position, i.e., positions 490 mm and 790 mm from the head side (see FIG. 2).

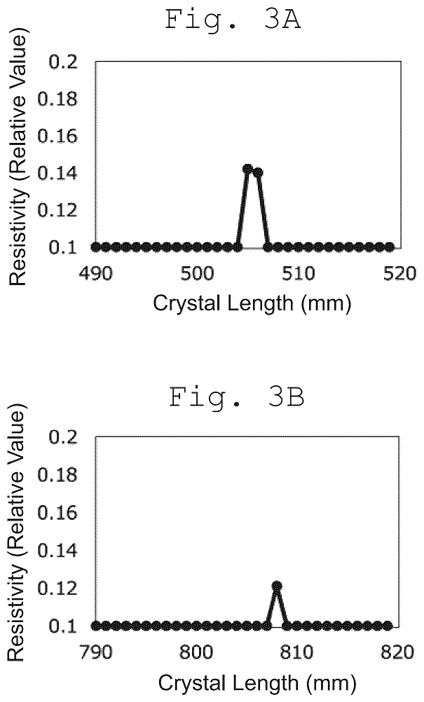

FIGS. 3A and 3B are viewgraphs showing the measurement results of the resistivity (relative values) on the side surface of a single-crystal ingot; FIG. 3A shows a change in relative resistivity in crystal length direction from a position at 490 mm from the head side as a start point, and FIG. 3B shows a change in relative resistivity in crystal length direction from a position at 790 mm from the head side as a start point. The resistivity measurement on the side surface of the single-crystal ingot by the four-point probe method at 30 points with a separation of 1 mm shows that the relative resistivity increases sharply near the sub-dopant added position; the maximum peak height is 42% at the position shown in FIG. 3A and 21% at the position shown in FIG. 3B, respectively. Thereafter, the relative resistivity decreased abruptly and became stable at the predetermined resistivity.

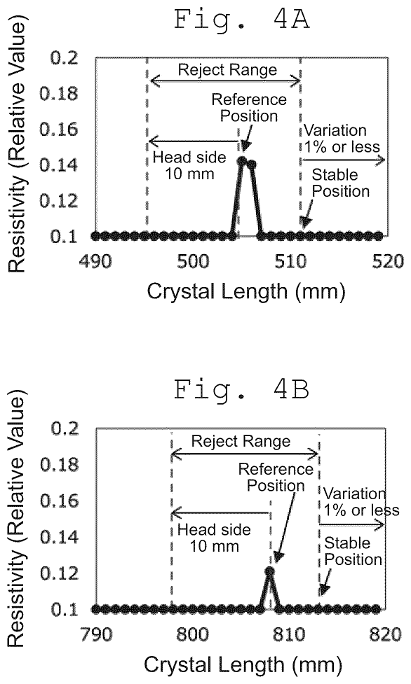

Near the position of 500 mm in the crystal length direction, the peak position with 42% of the change in the resistivity variation in the crystal length direction is defined as a reference position and, a position where the resistivity variation stabilizes and the variation of the relative resistivity in the crystal length direction becomes 1% or less and starts to depend on the segregation is defined as a stable position. Then, as shown in FIG. 4A, the reject range (16 mm) is defined as a range from a position 10 mm back to the head side from the reference position to the stable position. Further, near the position of 800 mm in the crystal length direction, the peak position with 21% of the change in the resistivity variation in the crystal length direction is defined as a reference position and, a position where the resistivity variation stabilizes and the variation of the relative resistivity in the crystal length direction becomes 1% or less and starts to depend on the segregation is defined as a stable position. As shown in FIG. 4B, the reject range (15 mm) is defined as a range from a position 10 mm back to the head side from the reference position to the stable position.

### Verification 1

Next, as described above, after measuring the resistivity on the side surface of the single-crystal ingot, wafers in the reject range described above are obtained by processing the block in the reject range with a separation of 1 mm to wafers. Then, the effects on the evaluation of resistivity variation on the side surface of the single-crystal ingot are verified by calculating the oxygen concentration and the number of thermal donors of the respective wafers. The oxygen concentration at the resistivity measurement positions was 0.55E18/cm³, the number of thermal donors obtained from the resistivity before and after donor killing was 3.9E13/cm³, and the variation in thermal donors in the single-crystal length direction was 3.18E12/cm³. From the results described above, it is confirmed that the number of thermal donors is the quantity that allows the evaluation of the peak value of the resistivity on the side surface of the single-crystal ingot shown in FIGS. 2A and 2B.

### Verification 2

Among the whole wafers processed in Verification 1 described above, wafers from the position of 490 mm to the position of 519 mm in the crystal length range and wafers from the position of 790 mm to the position of 819 mm in the crystal length range were taken and the resistivity at the center and the periphery of each taken wafers was measured by the four-point probe method.

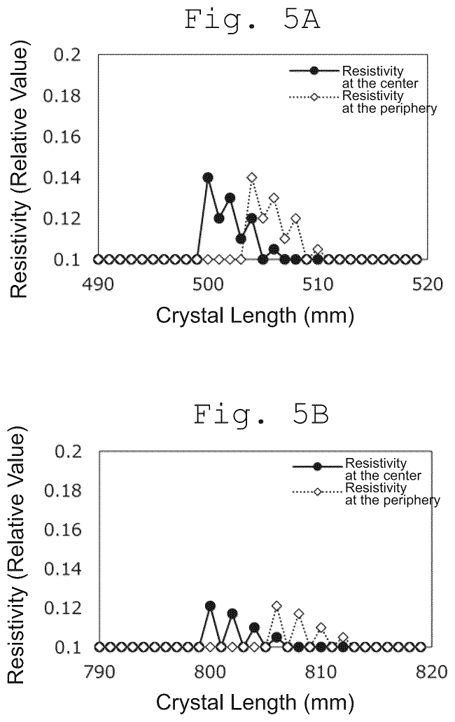

FIGS. 5A and 5B are viewgraphs showing the measurement results of resistivity (relative values) at the center and the periphery of the wafers; FIG. 5A shows the relative resistivity change of thirty (30) wafers taken from the range from the position of 490 mm to the position of 519 mm in the crystal length, and FIG. 5B shows the relative resistivity change of thirty (30) wafers taken from the range from the position of 790 mm to the position of 819 mm in the crystal length. As shown in FIGS. 5A and 5B, a rise in relative resistivity (resistivity increasing) occurs at the center of the wafers at the positions where the sub-dopant was added in both cases. In addition, in cases of both FIGS. 5A and 5B, an increase in the relative resistivity (resistivity increase) occurs at the outer circumference of the wafer, a few millimeters tail side of the peak position at the center of the wafer. From these results, it was confirmed that the resistivity variations on the side of the single-crystal ingot and the resistivity variations at the outer circumference of the wafer are generally consistent. In addition, as shown in FIGS. 5A and 5B, the peak positions of the wafer outer circumference are shifted 4 to 6 mm toward the tail side of the peak positions of the wafer center; as a result, this verification confirms the need to reject wafers from “the position 10 mm to the head side from the reference position” determined in Example 1.

### Verification 3

Next, the resistivity in the radial direction of each processed wafer was measured.

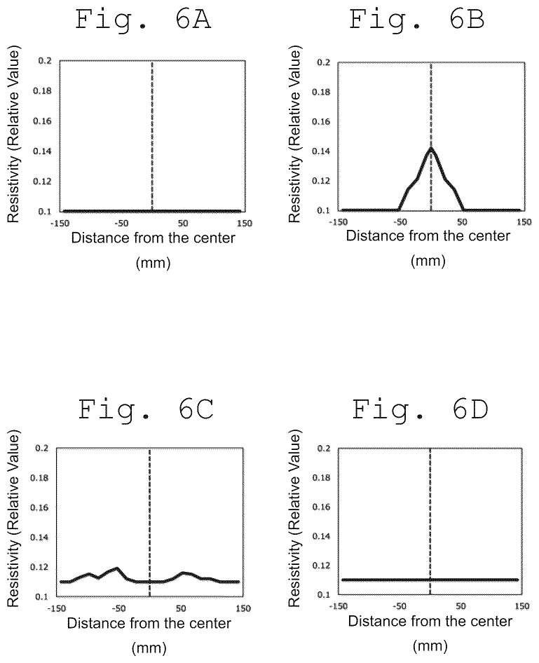

FIGS. 6A to 6D are views showing the measurement results of the resistivity (relative value) in the radial direction of wafers at positions indicated by a to d shown in FIG. 2, for example. FIG. 6A shows the variation of the relative resistivity in the radial direction at position a, FIG. 6B shows the variation of the relative resistivity in the radial direction at position b, FIG. 6C shows the variation of the relative resistivity in the radial direction at position c, and FIG. 6D shows the variation of the relative resistivity in the radial direction at position d. In Verification 3, the resistivity was measured for each wafer at an interval of 5 mm in the radial direction. As a result, for example, the relative resistivity is generally constant for the wafer at position a, which is before the sub-dopant is added to the crystal (see FIG. 6A), and the radial resistivity gradient (RRG) is generally constant at 5% or less. Note that RRG is a value, expressed as a percentage, obtained by dividing the difference between the maximum and minimum values in a group of resistivity measurements taken at any point within a single silicon crystal substrate by the minimum value. For example, the relative resistivity increases in the center of the wafer that is sliced at position b, where the sub-dopant is added (see FIG. 6B), and the RRG deteriorates (the RRG exceeds 5%). For example, for the wafer sliced at position c, which is after the sub-dopant is added, the relative resistivity increases at the outer periphery (see FIG. 6C) and the RRG remains in a state greater than 5%. For example, for the wafer sliced at position d which is after the sub-dopant is added to the far tail side, the relative resistivity in the radial direction does not increase (see FIG. 6D), and the relative resistivity stabilizes and the RRG improves to 5% or less.

By measuring the resistivity in the radial direction of the processed wafer described above, the position in the crystal length direction at which the RRG stabilizes again within 5% after the RRG deteriorates to exceed 5% can be specified. That is, the need to reject the portion up to the “stable position” determined in Example 1 is confirmed by the verification. In addition, the verification confirms that the RRG is within 5% for all of the wafers sliced from outside the reject range.

### Example 2

A lightly doped n-type counter doped crystal was pulled up under the same conditions as in Example 1, except that the desired oxygen concentration was 1.20E18/cm³.

Then, a single-crystal ingot was produced as shown in FIG. 2 by making the diameter 300 mm by peripheral grinding, and cutting the head and the tail cone parts. Then, the resistivity on the side surface in the crystal length direction was measured by the four-point probe method in a single-crystal ingot state, similar to Example 1 (see FIG. 2).

As a result, also for the single-crystal ingot of Example 2, the relative resistivity increased sharply near the sub-dopant added positions of 500 mm and 800 mm in crystal length, similar to Example 1, and then, the relative resistivity decreased sharply and stabilized to the desired resistivity.

In addition, also in Example 2, the reject range is defined as a range from a position 10 mm back to the head side from the reference position (the position where the increase of the relative resistivity in the crystal length direction increases to 10% or more) to the stable position (the position where the variation rate of the relative resistivity in the crystal length direction becomes 1% or less and starts to depend on the segregation).

Since the single-crystal ingot in Example 2 had a desired oxygen concentration of 1.20E18/cm³ and an amount of the variation of the thermal donor of 7.21E12/cm³, the reference position (the crystal length position where the sub-dopant was added) was detected by the resistivity variation of 12% or more.

### COMPARATIVE EXAMPLE

In the Comparative Example, a lightly doped n-type counter doped crystal was pulled up under the same conditions as Example 1, and a single-crystal ingot similar to Example 1 was produced.

In Comparative Example, the produced single-crystal ingot was cut into blocks and the resistivity evaluation (an evaluation of whether the RRG is within 5%) was performed on test sample wafers sliced at the time when the blocks were prepared (conventional method).

However, since it is not clear with this method at what mm position from the end of the block the resistivity increases (the RRG exceeds 5%), the position where the test sample wafers were sliced was at the 490 mm and 510 mm positions in the crystal length direction for the sub-dopant added position of 500 mm. After processing the respective test sample wafers and evaluating the resistivity, the RRG was found to be 3.4% for the 490 mm case and 7.0% for the 510 mm case in the crystal length direction. Table 1 shows the RRG results of the sliced samples.

TABLE 1

| Slicing Position of ID saw | RRG | Determination |
|---|---|---|
| 490 mm | 3.4% | Good |
| 510 mm | 7.0% | Not Good |

Thus, the resistivity evaluation was carried out with test sample wafers sliced at a constant thickness by the wire saw. Since the resistivity evaluation for a test sample wafer sliced at a position of 511 mm in the crystal length direction and subjected to processing resulted in an RRG of 5.2%, again, the resistivity evaluation for a test sample wafer sliced at a position of 512 mm in the crystal length direction and subjected to processing resulted in an RRG of 3.9%. The kerf loss in crystal length due to slicing the block and processing was 22 mm.

TABLE 2

| Slicing Position of | | |
|---|---|---|
| Wire saw | RRG | Determination |
| 511 mm | 5.2% | Not Good |
| 512 mm | 3.9% | Good |

Meanwhile, in Examples 1 and 2, as described above, the reject range can be determined immediately by specifying the high resistivity range from the resistivity distribution on the side surface of the single-crystal ingot, and, after the rejection the wafer processing can be performed using the silicon block having the RRG of 5% or less. This significantly improves the wafer yield (possibly approximately 30% on average) compared to the method (conventional one) in which test sample wafers are sliced from the single-crystal ingot and the resistivity evaluation is performed. In addition, the time required for resistivity evaluation was able to be significantly reduced compared to the conventional method (possibly an average of 12 hours or so).

Note that as another comparative example, there is a method in which a lightly doped n-type counter dope crystal is pulled up under conditions similar to Example 1, and a block is cut at a sub-dopant added position in the crystal length direction based on the log data. However, a problem arose that a block at the sub-dopant added position could not be cut due to ambiguity of the sub-dopant added position due to the peripheral grinding of the single-crystal ingot and the slicing process of wafers.

The present invention is not limited to the above embodiments and examples. The above embodiments and examples are illustrative examples, and any product having substantially the same configuration and similar effects as the technical concept described in the claims of the present invention is included within the technical scope of the present invention.

### INDUSTRIAL APPLICABILITY

As described above, the silicon single-crystal ingot evaluation method is useful for silicon single-crystal ingots produced by the Czochralski method (CZ method) and is particularly suitable for silicon single-crystal ingots to keep wafer resistivity within a desired range.

### LIST OF REFERENCE SIGNS

1 single-crystal ingot (silicon single-crystal ingot)

1. A method for evaluating a silicon single-crystal ingot that has been counter doped by adding a sub-dopant during the pulling of silicon single-crystals ingot by the CZ method, the method comprising:
a step of measuring the resistivity in a crystal length direction by a four-point probe method on a side surface in a silicon single-crystal ingot state; and a step of determining a reject range on the basis of a resistivity distribution in the crystal length direction.

2. The method for evaluating a silicon single-crystal ingot according to claim 1, wherein a reference position is defined as a position at which the peak of the resistivity is higher than 10% or more compared to the resistivity of a stable region of a head side where the resistivity variation is stable, and the reject range is defined as a range between positions that are separated from the reference position toward the head side and the tail side by predetermined lengths.

3. The method for evaluating a silicon single-crystal ingot according to claim 1, wherein a reference position is defined as a position at which the peak of the resistivity is higher than 10% or more compared to the resistivity of a stable region of a head side where the resistivity variation is stable, and further, a stable position is defined as a position at which the resistivity variation is stable on a tail side from the reference position and the resistivity variation is not more than a threshold value and starts to depend on segregation, and the reject range is defined as a range from a position of a predetermined length back to the head side from the reference position to the stable position.

4. The method for evaluating a silicon single-crystal ingot according to claim 1, wherein a resistivity measurement range includes at least a range from the sub-dopant added position to the position where the resistivity increases and then stabilizes to the desired resistivity again.

5. The method for evaluating a silicon single-crystal ingot according to claim 1, wherein the resistivity of a silicon single-crystal ingot to be grown is 10 Ω·cm or more.

6. The method for evaluating a silicon single-crystal ingot according to claim 2, wherein the reject range is defined as a range from the reference position 8 mm or more to 12 mm or less toward the head and 10 mm or more to 20 mm or less toward the tail.

7. The method for evaluating a silicon single-crystal ingot according to claim 3, wherein the reject range is defined as a range from the reference position 8 mm or more to 12 mm or less toward the head and 10 mm or more to 20 mm or less toward the tail.
